(19) United States
(12) Patent Application Publication
(10) Pub. No.: US 2008/0154168 A1
(43) Pub. Date: Jun. 26, 2008

(54) SURGICAL BANDAGE FOR USE WITH TISSUE ADHESIVES AND OTHER MEDICAMENTS

(76) Inventor: Thomas Placido Lutri, New York, NY (US)

Correspondence Address: PROSKAUER ROSE LLP, ONE INTERNATIONAL PLACE, BOSTON, MA, 02110, US

(21) Appl. No.: 11/963,760

(22) Filed: Dec. 21, 2007

Related U.S. Application Data
(63) Continuation-in-part of application No. 11/881,395, filed on Jul. 26, 2007.
Application No. 11/881,395 is a continuation of application No. 10/621,567, filed on Jul. 17, 2003, now abandoned.

(51) Int. Cl. A61F 13/02

(52) U.S. Cl. 602/54

(57) ABSTRACT

Disclosed is a bandage, and methods for its use, which facilitates application of tissue adhesive to close and seal wounds.

### RELATED APPLICATIONS

This application is a Continuation-in-Part of U.S. application Ser. No. 11/881,395, filed Jul. 26, 2007 which is a Continuation of Ser. No. 10/621,567, filed Jul. 17, 2003, the entire teachings of each of which are incorporated herein by reference.

### FIELD OF THE INVENTION

This invention relates to surgical bandages and to medical and surgical wound apposition and closure devices which may involve the use of tissue adhesives.

### BACKGROUND

Tissue adhesives have recently been developed which achieve wound closure by bonding wound edges in close apposition, while the natural process of wound healing occurs. These tissue adhesives include both cyanoacrylate and fibrin based materials. Cyanoacrylates form a solid mass that bridges wound edges at their surfaces, thereby closing the wound. Especially with cyanoacrylates, it is critically important that adhesive not pass into the wound itself. Quinn et. al. (1997). Thus, special care must be taken to ensure that wound edges are kept in close apposition for the amount of time required to form a solid bridge of adhesive material. A total of 3-4 applications must be spread in layers directly over the junction of tightly apposed wound edges, allowing 10-15 seconds between applications for the adhesive to harden. Wound edges must be kept in close approximation for about 2 and ½ minutes. Better cosmetic results (i.e., less scarring) are obtained after healing when wound edges are kept in close apposition.

Typically, during application of tissue adhesive, wound edges are held in apposition by gloved fingers or forceps. Quinn et al. (1997). This manual apposition of wound edges is awkward, requires technical skill, and is susceptible to disruption if the patient moves during the process. Thus, it would be of considerable utility to simplify the process of tissue adhesive application, not only for health care professionals but for lay consumers. Clark et. al. (1993 and 1995) developed wound closure devices which achieve apposition of wound edges during application of tissue adhesive. These devices employ a porous “bonding pad” which acts as a matrix for adhesive. These devices have the disadvantage of not allowing direct visualization of the wound and the adjacent areas before, during and after application of adhesive. Direct visualization during the process of wound closure is critical in achieving optimal cosmetic results. In addition, the devices by Clark et. al. do not allow direct contact of fluid to the wound surface through at least one unobstructed fluid path. Nor do they allow blood, transudate or other fluid in or near the wound to be wiped away and/or blotted dry prior to application of any substance. Removal of blood and transudates further aids in visualization. Removal of blood and other fluids is also important because they serve as media for bacterial growth, and are thus a source of infection.

### SUMMARY OF THE INVENTION

The present invention is a surgical bandage comprised of an adhesive strip with an opening or openings through which tissue adhesive may be applied to a wound, laceration, surgical incision or other tissue separation. The adhesive strip can be a single continuous piece, with a window or opening or plurality of openings which provide direct visualization and access to a wound. Alternatively, the surgical bandage can be formed by two adhesive end segments connected in a linear fashion to a central bridging segment which provides an opening or openings through which a wound and adjacent surrounding tissue surfaces may be visualized and tissue adhesive applied. Each end segment can include two or more appendages that may provide useful advantages in certain situations including location and/or size of the wound, laceration, surgical incision or tissue separation. The surgical bandage can be attached to a patient's skin in a manner which brings the wound edges into close approximation, temporarily closing the wound. Tissue adhesive or liquids or other substances can then be applied directly to the temporarily closed wound through the opening or openings or central bridging segment of the surgical bandage.

Provided herein are surgical bandages. In some embodiments, the surgical bandage comprises an adhesive strip containing an opening. The bandage is configured such that the strip can be adhered to the tissue surface in a manner that closes a tissue separation, such that the opening extends across the closed tissue separation and fluid can be applied to the closed tissue separation through the opening. In some embodiments, at least a portion of the bandage is made from or coated with a material that is non-adherent to a fluid adhesive upon setting of the fluid adhesive.

In some embodiments, the surgical bandage comprises a central bridging portion and two segments, wherein the closed wound is contained within the central bridging portion and wherein the central bridging portion is made from or coated with a material that is non-adherent to a fluid adhesive upon setting of the fluid adhesive. In some embodiments, the two segments are made from or coated with a material that is non-adherent to a fluid adhesive upon setting of the fluid adhesive. In some embodiments, one face of the bandage is coated with an adhesive and another face of the bandage is coated with the material that is non-adherent to a fluid adhesive upon setting of the fluid adhesive. In some embodiments, the end segments and central bridging segment are formed from different material.

Methods of closing a wound are also provided. In some embodiments, the methods comprise affixing one end of a surgical bandage provided herein to one side of the open wound, pulling on another end of the surgical bandage in a manner to close at least a portion of the open wound, affixing the other end of the surgical bandage to the other side of the closed wound, and dispensing a fluid tissue adhesive through the opening, wherein the fluid flows through the opening to directly contact the closed wound. Where the wound is larger or longer, for example 5 inches or more in one dimension, two or more bandages provided herein can be used to close the wound. In some embodiments, where more the one bandage is used, the bandages can be affixed to the tissue such that the edges of one or more adjacently placed bandages are touching. In other embodiments, where more the one bandage is used, the bandages can be affixed to the tissue such that the adjacent bandages are spaced apart from each other.

Wound closure kits are also provided herein. In some embodiments the kits comprise the surgical bandage provided herein and a source of fluid tissue adhesive. In some embodiments the fluid tissue adhesive is, for example, cyanoacrylate or enhanced viscosity cyanoacrylate.

The various embodiments described herein can be complimentary and can be combined or used together in a manner understood by the skilled person in view of the teachings contained herein.

### BRIEF DESCRIPTION OF THE DRAWINGS

FIG. 1 is a top view of one embodiment of the surgical bandage according to the present invention.

FIG. 2 is a top view of one embodiment of the surgical bandage according to the present invention.

FIG. 3 is a side view of one embodiment of the surgical bandage according to the present invention.

FIG. 4 is a top view of one embodiment of the surgical bandage according to the present invention.

FIG. 5 is a top view of one embodiment of the surgical bandage according to the present invention.

FIG. 6 is a top view of one embodiment of the surgical bandage according to the present invention.

FIG. 7 is a top view of one embodiment of the surgical bandage according to the present invention.

FIG. 8 shows the surgical bandage of the present invention where each end segment has two appendages.

FIG. 9 shows the surgical bandage of the present invention where each end segment has three appendages.

FIG. 10 shows an oblique view of one embodiment of the surgical bandage according to the present invention.

FIG. 11 is a top view of a tissue surface containing a tissue separation and the surgical bandage of the present invention, showing an initial step in the process of using the surgical bandage.

FIG. 12 is a top view of a tissue surface containing a tissue separation and the surgical bandage of the present invention, further demonstrating the process of using the surgical bandage.

FIG. 13 is a top view of a tissue surface containing a closed tissue separation and the surgical bandage of the present invention, further demonstrating the process of using the surgical bandage.

FIG. 14 is a top view of a tissue surface containing a closed tissue separation and the surgical bandage of the present invention, further demonstrating the process of using the surgical bandage.

FIG. 15 is a top view of a tissue surface containing a closed tissue separation and the surgical bandage of the present invention, further demonstrating the process of using the surgical bandage.

FIG. 16 shows the surgical bandage of the present invention after the two end segments have been separated and removed.

### DETAILED DESCRIPTION

In embodiments containing a single continuous piece, the surgical bandage is formed from tape with adhesive on one side. An opening or openings in the tape material permits direct access to a wound, laceration, surgical incision or other tissue separation. The bandage may be packaged as a continuous roll with alternating tape end segments and central bridging segments.

The tape should ideally be non-allergenic and non-irritating to humans and animals. Materials used to make the tape can be absorbent or non-absorbent and can include any suitable material including thin plastic; polymers such as polyvinyl, polypropylene, polyurethane or polyester; fabrics (such as cotton, nylon, silk or other naturally occurring or synthetic fabrics), bio-absorbable materials including those used to manufacture absorbable sutures, silicon or silicon coated material, latex or rubber, Teflon and Teflon related products, acetate products, Kevlar and paper, cellulose or fiber-based material. In some embodiments, the material used to make the tape is non-adherent to tissue adhesives such as cyanoacrylate or is coated with a substance that renders the material non-adherent to tissue adhesives such as cyanoacrylate. In some embodiments, the material that is non-adherent to tissue adhesives such as cyanoacrylate can be, for example, polyethylene, polyester, silicone, or acetate products such as ethylene vinyl acetate (EVA). In some embodiments a portion of the bandage is coated with a substance that renders the material non-adherent to tissue adhesives such as cyanoacrylate. The non-coated portions of the bandage are expected to have higher breathability compared to the coated portion(s) of the bandage. In some embodiments, the central bridging portion of the bandage is coated with a substance that renders the central bridging portion non-adherent to tissue adhesives such as cyanoacrylate.

In embodiments containing a central bridging segment, two adhesive end segments can be formed either from tape or other suitable material, with adhesive on at least one side. Embodiments comprising a central bridging segment may optionally provide a means for elevating the central bridging segment above the tissue surface. Alternatively, the central bridging segment could be-elevated by pre-applying two thickened bridge supports, with adhesive on at least one side, on tissue surface adjacent to the wound. Then, using one of the embodiments of the surgical bandage to span the space between the two supports, a bridge is formed above the wound, laceration, surgical incision or tissue separation. The adhesive side of the end segments or optional bridge supports can have a separate protective layer which must be peeled away before the tape can be applied to an appropriate surface. This protective layer prevents the tape from accidentally sticking to an unintended surface and also helps maintain adhesiveness of the tape.

The opening is of suitable size to expose the closed wound after the bandage has been applied to the tissue to close the wound. In some embodiments, the opening of the bandage is configured such that when the bandage is applied to close a wound, at least 80 percent of the closed wound can be visualized through the opening. In some embodiments, the opening is configured such that at least 90 percent of the closed can be visualized through the opening.

An optional “pull string” string or thread-like device can be placed within or under the tape end segments at-the border where the tape meets the opening or central bridging segment. This facilitates cutting, separating and removing tape end segments in a manner similar to the operation of a “pull-string” in a FedEx® envelope. Means for detachment enables the tape end segments to be removed after the wound is closed by hardened adhesive.

The bandage itself and/or the central bridging segment can be made from transparent materials to permit better visualization of the wound.

In embodiments with end segments and a central bridging segment, the bridging segment can have an opening or a plurality of openings in the material of which the end segments are formed. Alternatively the bridging segment can be formed from strands or fibers, arranged in parallel or otherwise, which permit visualization of the wound and direct access for application of tissue adhesive or medicaments. Materials used to make the strands or fibers for the central bridging segment can include any suitable material, including the materials described above used to make tape segments, including bio-absorbable materials used to manufacture absorbable sutures. These strands or fibers can also be made from materials that may or may not be coated with another substance such as resin, wax, silicon, plastic or other polymer, latex or other rubber, or other coating substance. The opening or openings, including the spaces between strands or fibers, will also allow any blood, transudate or other fluid that might accumulate in or near the wound to be expressed, wiped away or blotted dry. The adhesive end segments can also include a suitable “thickening” material or otherwise provide means of elevating the central bridging segment above the tissue surface. The central bridging segment may or may not have adhesive on one side.

The opening or openings of the surgical bandage allow direct access to the wound for medicines or other agents which promote wound healing, in addition to tissue adhesive. The opening or openings can be of any shape selected from the various geometric, circular or irregular shapes or be any combination thereof. Once an appropriate amount of adhesive has been applied and sufficient time has elapsed to allow the adhesive to harden or set, excess tape, or the two end segments, can be removed using the optional “pull-string” described previously. Alternatively, the bandage can be left in place, intact, after the tissue adhesive has dried, to add extra “holding power” or tensile strength during the natural wound healing process. Both the dried adhesive and the bandage in this way continue to hold the wound edges together. Alternatively, where the bandage is made from or coated with a material that is non-adherent to the tissue adhesive such as cyanoacrylate (e.g., made from or coated with a polyethylene, a polyester, an acetate product or with silicone), the bandage will be able to fall off of the wound, leaving the tissue adhesive adhered to the wound, thereby allowing the tissue adhesive to continue to hold the wound edges together.

Use of the Surgical Bandage

In referring to a skin surface containing a wound, laceration or surgical incision, this explanation of the use of the surgical bandage is for illustrative purposes and does not limit the use of the bandage in other types of tissue separations. The bandage of the present invention can be used either in humans or non-human animals. Prior to using the bandage, a wound, laceration, surgical incision or other tissue separation should be cleaned and debrided in manner consistent with accepted medical or veterinary practice. If necessary, any layered closure using subcutaneous or deep sutures should be done prior to use of the surgical bandage. The area around the wound, laceration or surgical incision should be made dry to ensure good adhesion of the surgical bandage. Immediately prior to placement of the surgical bandage a substance that may improve the adhesiveness of the surgical bandage such as tincture of benzoin may be applied to the surrounding skin. Optionally, the bandage may contain a protective layer which must be removed in order to expose the adhesive side of the tape.

One end of the tape or one end segment is placed with the bare adhesive surface facing toward the skin surface adjacent to one side of the gaping wound, laceration or surgical incision and is pressed securely onto the skin surface so that adhesion occurs. The tape segment should be placed so that the opening, openings or central bridging segment directly overlies and spans the gaping wound, laceration or surgical incision Once one side of the surgical bandage has been placed and is adherent, the apposing wound edges should be brought into close approximation by pulling, with appropriate tension or traction, on the end that has not yet been placed on or adhered to the skin surface. Because direct visualization is possible through the opening or openings or central bridging segment, the alignment of the edges of the wound, laceration or surgical incision can be accomplished in an optimal manner.

Once the alignment is optimal or acceptable, the free tape end or end segment is placed and pressed onto the skin surface on the other side of the wound, laceration or surgical incision. At this point the gap previously present in the wound, laceration or surgical incision should be significantly reduced or absent, and its edges should be held securely together in close approximation by the surgical bandage. Because of design features of the bandage, the process of applying the surgical bandage helps achieve hemostasis. In addition, the opening or openings in the bandage allow blood and fluids to be easily expressed, wiped away and/or blotted dry, which is beneficial in removing a potential source of infection.

A medicine or an agent to promote wound healing can be applied at this time. Such agents may be aerosolized fluids, antibiotics, anesthetic, gels, lotions, liquid water solutions such as sterile saline, tissue sealants, creams, ointments or other wound healing, and combinations thereof.

After the edges of the wound, laceration or surgical incision are securely held together in close approximation, a tissue adhesive may be applied. A cyanoacrylate or other adhesive can easily be applied to the appropriate areas of a temporarily closed wound using direct visualization and access through the opening or openings or central bridging segment. Such adhesives may include bio-adhesives, synthetic adhesives, enhanced viscosity cyanoacrylates, fibrin, or fibrin-like substances. Once the recommended amount of adhesive has been applied and sufficient time has elapsed to allow the adhesive to harden or set, excess tape or the two end segments can be removed using the “pull-string” previously described.

### Example 1

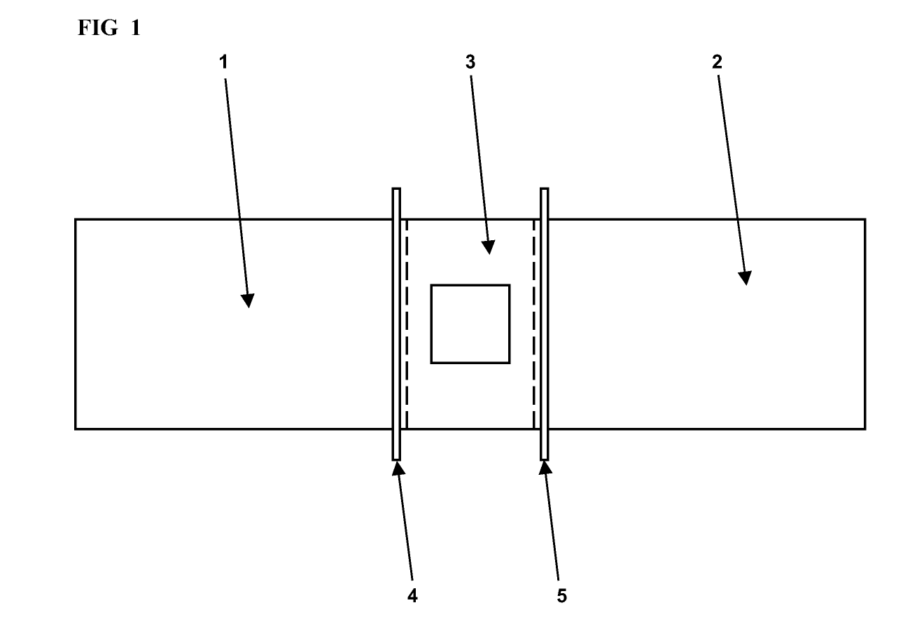

FIG. 1 is a top view of one embodiment of the surgical bandage provided herein. The surgical bandage is preferably packaged in a sterile condition within a package and distributed as a single-use surgical bandage. The surgical bandage includes two tape end segments, 1 and 2, a central bridging segment, 3, and two optional “pull strings,” 4 and 5. In this embodiment, the bandage is one continuous piece, with a single opening or “window” framed by tape material.

### Example 2

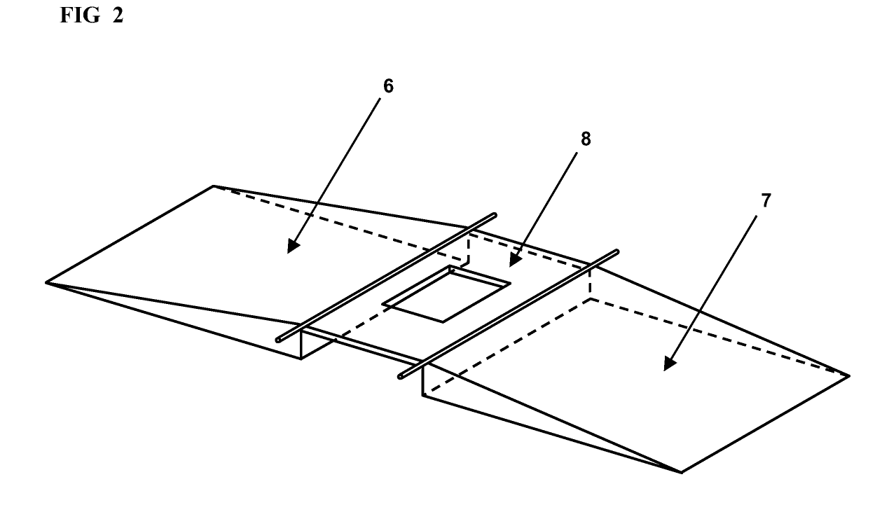

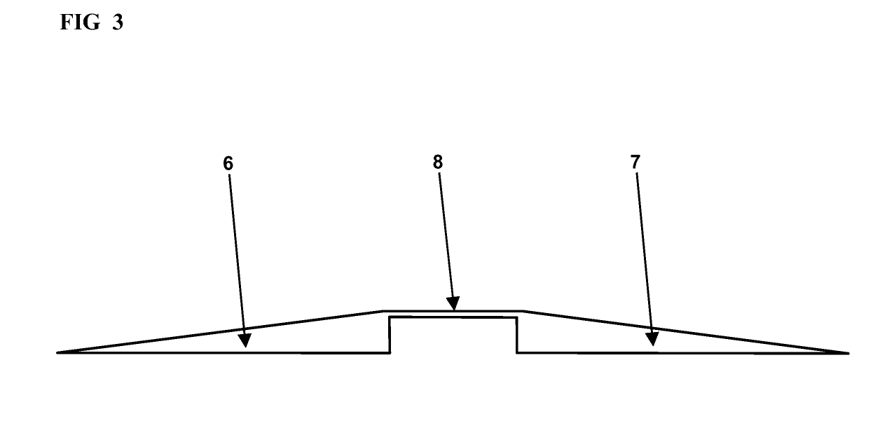

FIG. 2 is a top view and FIG. 3 a side view of another embodiment of the surgical bandage provided herein. The bandage includes two end segments, 6 and 7, which are thickened or wedge shaped to provide means of elevating the central bridging segment, 8, above the tissue surface.

### Example 3

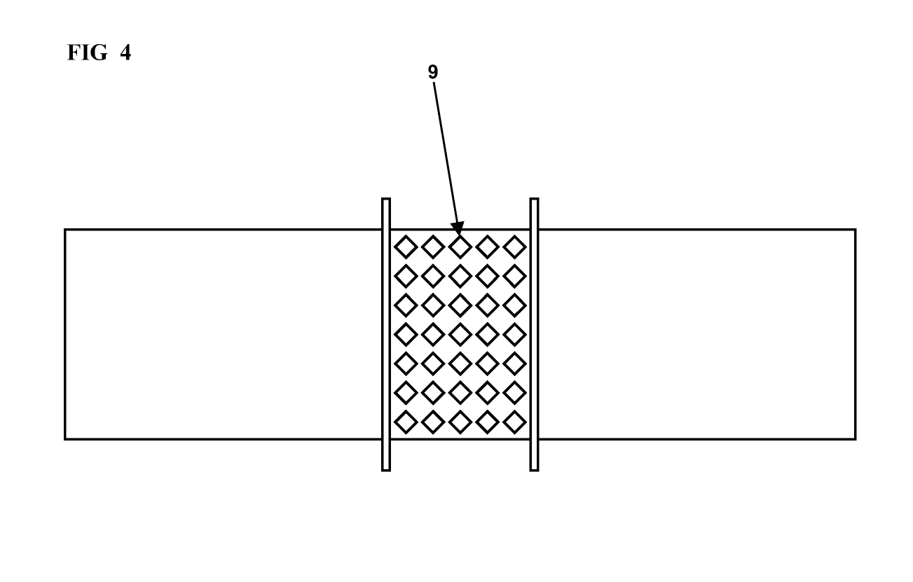

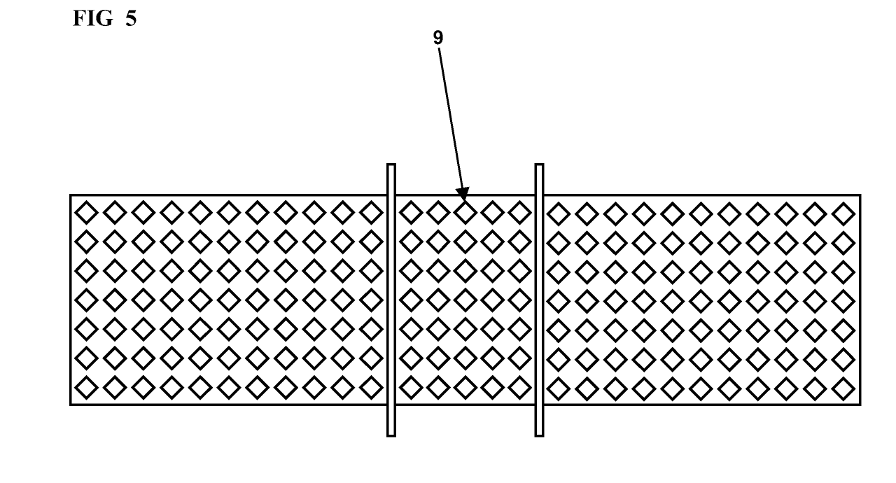

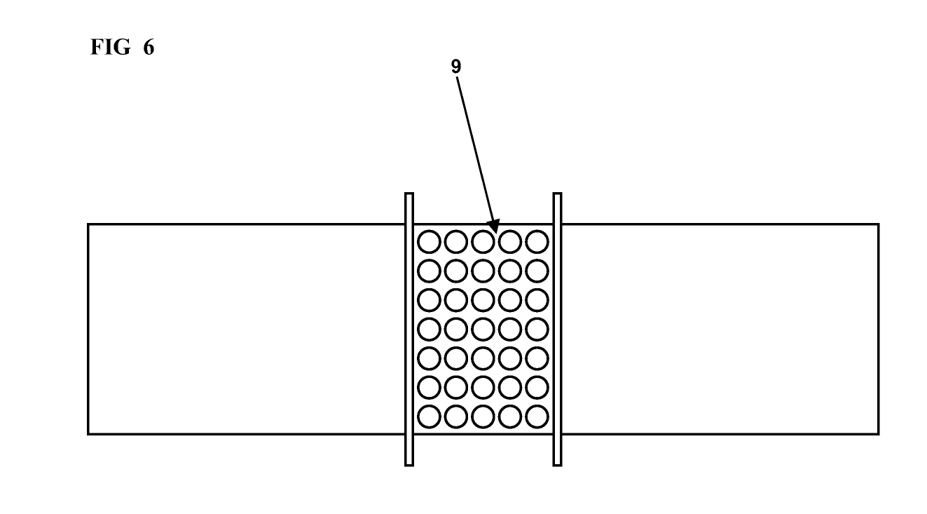

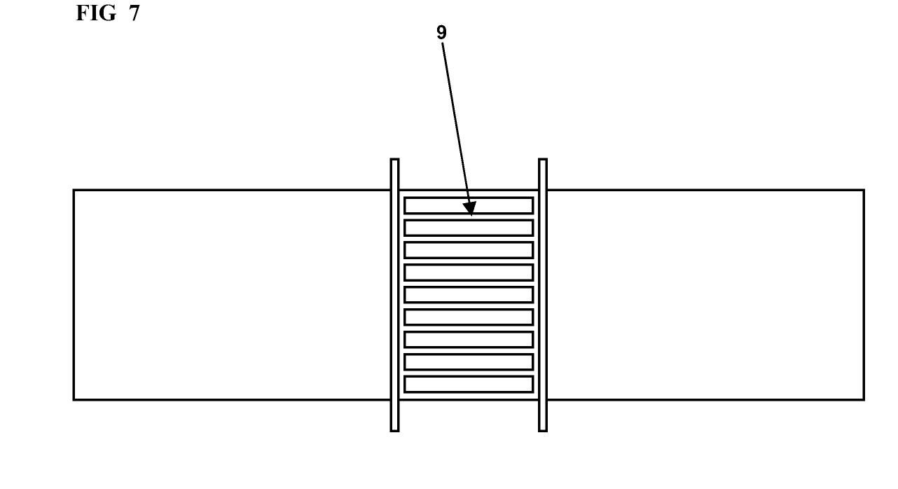

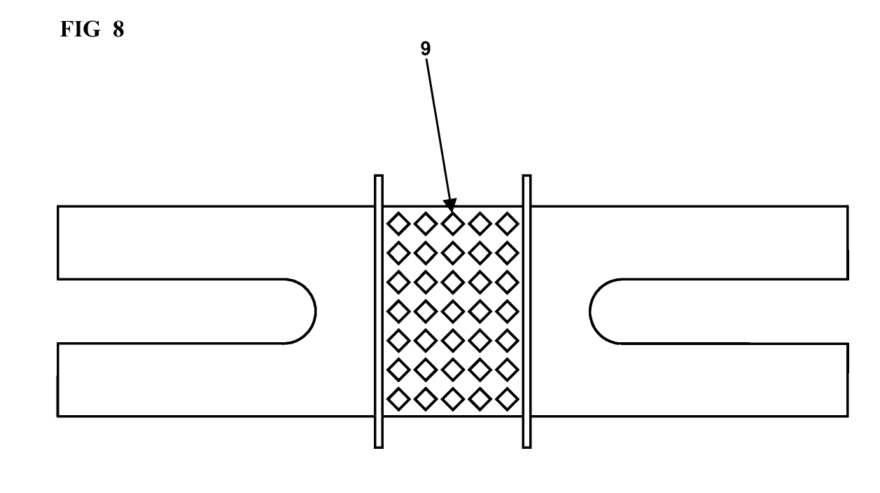

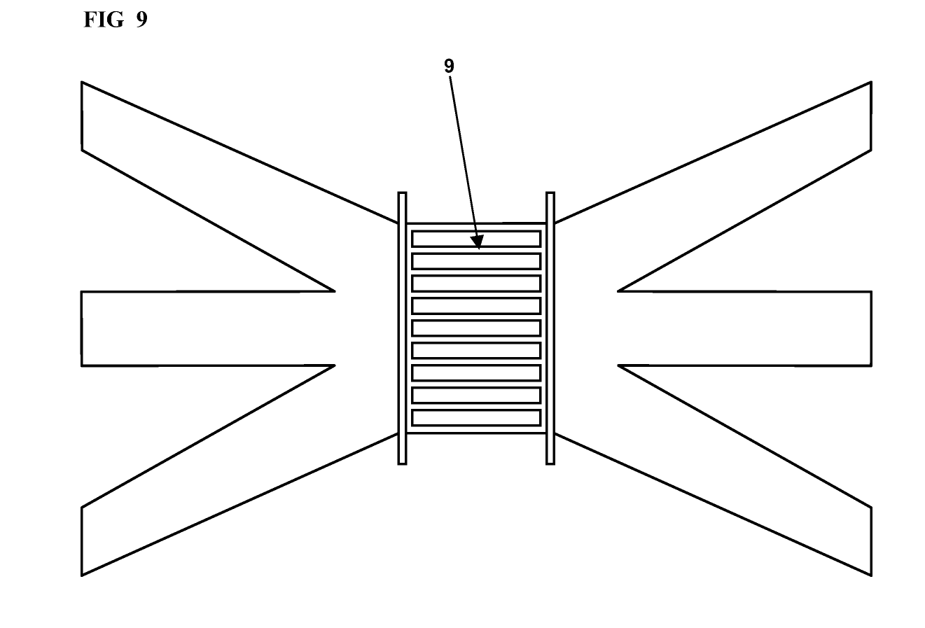

FIGS. 4 through 7 provide top views of other embodiments of the surgical bandage. The central bridging segments, 9, can be formed from strands or fibers, arranged parallel or otherwise. Alternatively, the central bridging segment, 9, can be formed from a thin layer with interstices or can consist of a plurality of openings in the tape material. FIGS. 8 and 9 depict alternate embodiments which have end segments with two or more appendages.

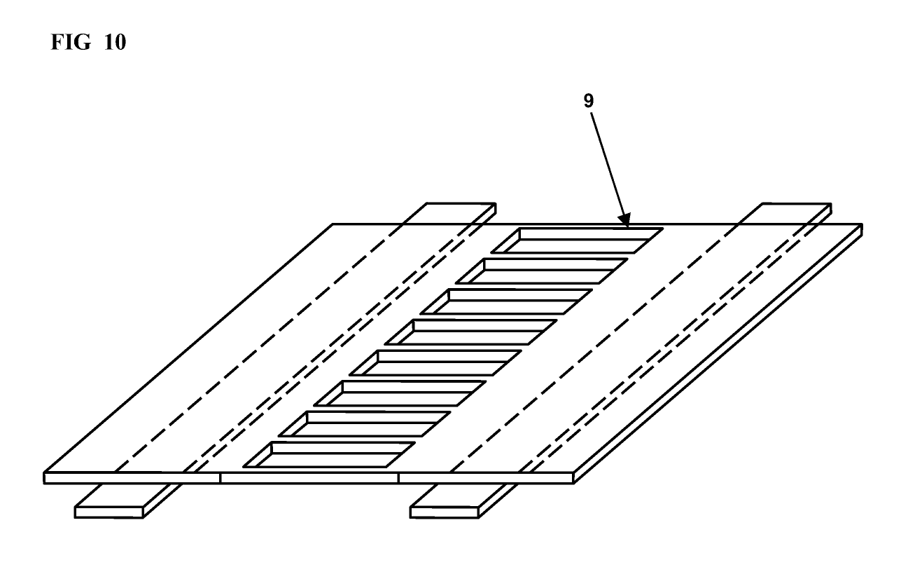

FIG. 10 is an oblique view of another embodiment showing an elevated central bridging segment, 18, supported by two separate bridge supports, 19 and 20.

### Example 4

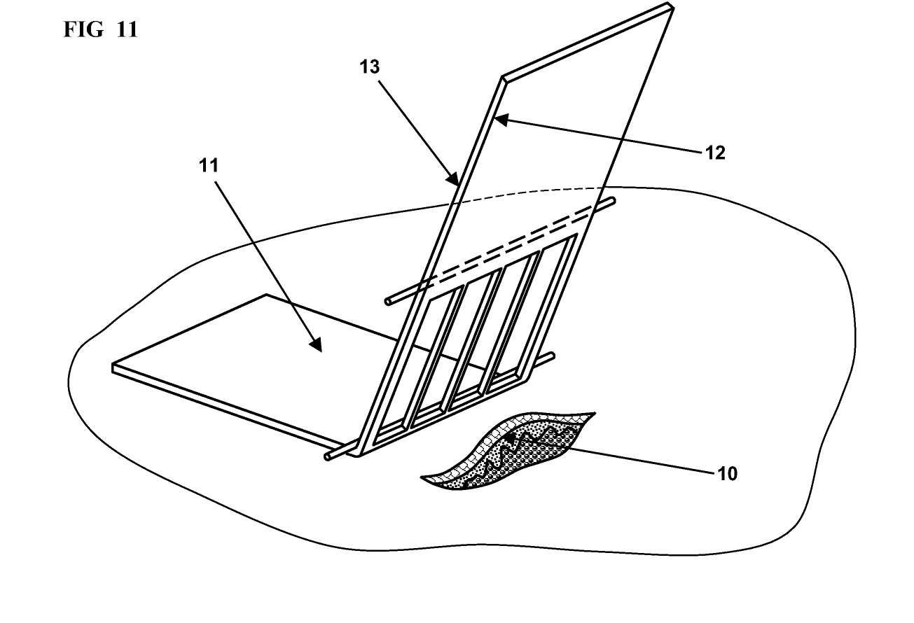

FIGS. 11 through 16 provide a visual demonstration of the use of the surgical bandage. FIG. 11 is a top view of a tissue surface that has a laceration, 10. In FIG. 11 one end segment, 11, has had its protective layer removed, and the tape segment, 11, has been pressed onto the skin surface on one side of a laceration, 10. The tape segment, 11, is adhering firmly to the skin surface in FIG. 11. The protective layer, 12, has not yet been removed from the tape end segment, 1-3.

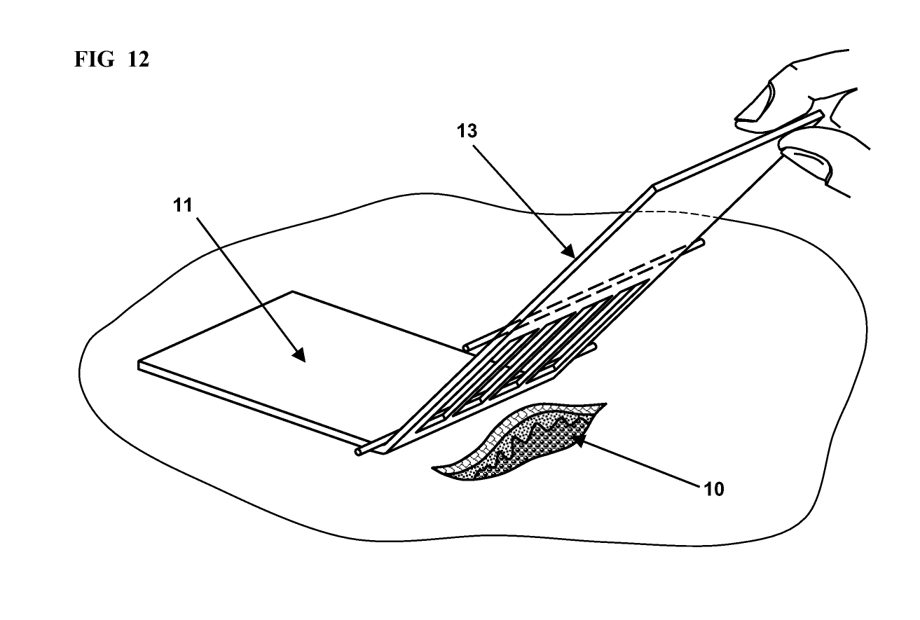

In FIG. 12 the laceration, 10, is shown to be gaping with the tape end segment, 11, adhering to the skin surface adjacent to one of the edges of the laceration, 10. In FIG. 12 the tape segment, 13, has also had its protective layer removed. The tape end segment, 13, is being grasped by the person applying the surgical bandage and is being held above the tissue surface.

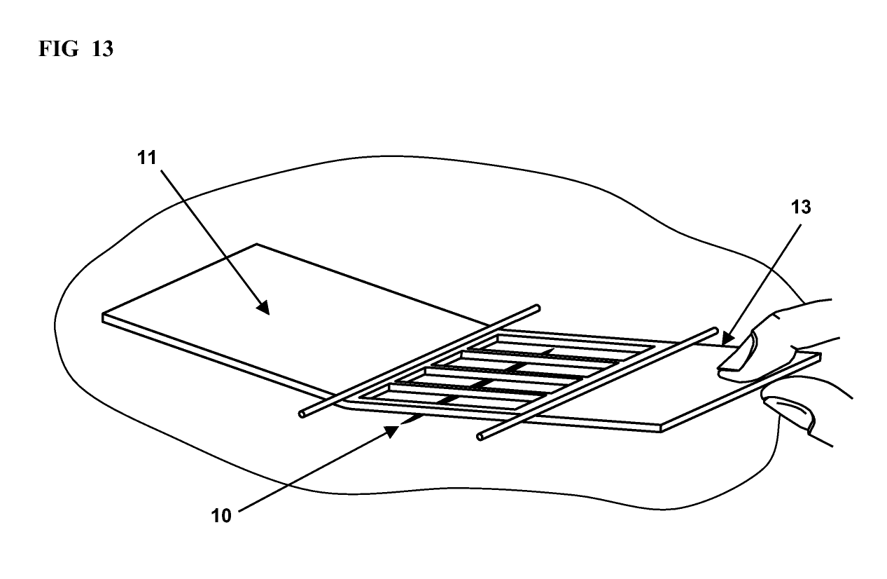

FIG. 13 shows that one end segment, 11 is firmly adherent to one side of the laceration, 10, and the person applying the surgical bandage is pulling or using traction on the unattached end segment, 13. In FIG. 13, the tension or traction applied to the unattached tape end segment, 13, has caused the wound edges of the laceration, 10, to come together and be closely approximated.

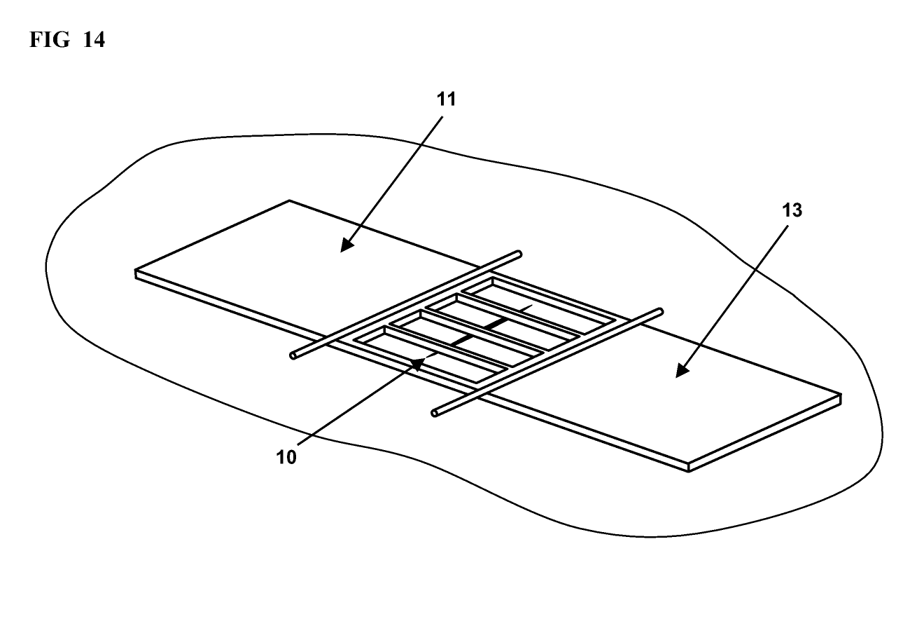

FIG. 14 shows that both end segments of tape, 11 and 13, have been pressed against and are adherent to the skin surface adjacent to the closely approximated laceration, 10. In FIG. 14, the central bridging segment, 14, is positioned against and spans the laceration, 10. FIG. 14 shows a closed wound, 10, to which medicaments and/or adhesives can be applied.

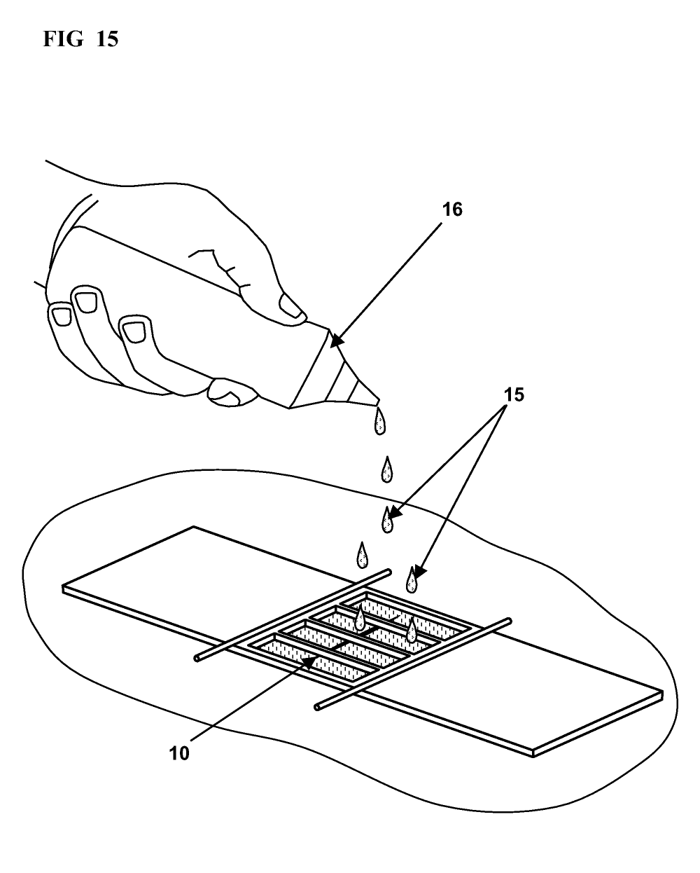

FIG. 15 shows the closely approximated laceration, 10, covered by the central bridging segment. In FIG. 15 a tissue adhesive, 15, from a tube, 16, is applied and allowed to harden.

### Example 5

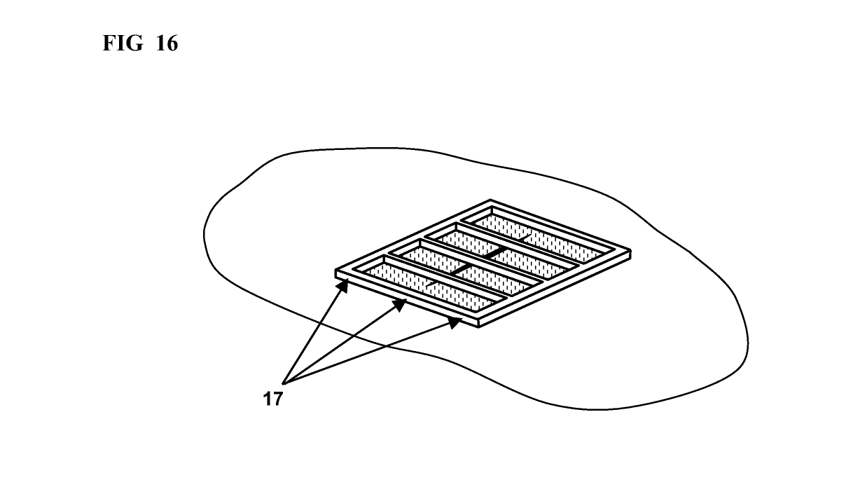

FIG. 16 shows the surgical bandage after the two end segments have been separated from the central bridging segment, 17, using the optional “pull strings.”

### Example 6

Preferably, the present invention can be distributed as a wound treatment kit having at least one bandage packaged in a sterile condition and a source of tissue adhesive.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

What is claimed is:

1. A surgical bandage comprising an adhesive strip containing an opening wherein the strip can be adhered to a tissue surface in a manner that closes a tissue separation, such that the opening extends across the closed tissue separation and fluid can be applied to the closed tissue separation through the opening, and wherein at least a portion of the bandage is made from or coated with a material that is non-adherent to a fluid adhesive upon setting of the fluid adhesive.

2. The surgical bandage of claim 1 packaged as a continuous roll with alternating tape segments and a segment containing the opening.

3. The surgical bandage of claim 1 wherein at least a portion of the bandage is made from silicone or coated with a silicone.

4. The surgical bandage of claim 1, wherein at least a portion of the bandage is made from ethylene vinyl acetate.

5. The surgical bandage of claim 1, wherein at least a portion of the bandage is made from polyethylene.

6. The surgical bandage of claim 1, further comprising a central bridging portion and two segments, wherein the opening is contained within the central bridging portion and wherein the central bridging portion is made from or coated with a material that is non-adherent to a fluid adhesive upon setting of the fluid adhesive.

7. The surgical bandage of claim 6, wherein the central bridging portion and the two segments are made from or coated with a material that is non-adherent to a fluid adhesive upon setting of the fluid adhesive.

8. The surgical bandage of claim 1, wherein one face of the bandage is coated with an adhesive and another face of the bandage is coated with the material that is non-adherent to a fluid adhesive upon setting of the fluid adhesive.

9. The surgical bandage of claim 1, wherein the opening is configured such that when the bandage is applied to close a wound, at least 80 percent of the closed wound can be visualized through the opening.

10. The surgical bandage of claim 1, wherein the opening is configured such that when the bandage is applied to close a wound, at least 90 percent of the closed can be visualized through the opening.

11. A method of wound closure comprising affixing one end of a surgical bandage of claim 1 to one side of the open wound, pulling on another end of the surgical bandage in a manner to close at least a portion of the open wound, affixing the other end of the surgical bandage to another side of the closed wound, and dispensing a fluid tissue adhesive through the opening, wherein the fluid flows through the opening to directly contact the closed wound.

12. The method of claim 11, wherein the tissue adhesive is an enhanced viscosity cyanoacrylate.

13. A wound closure kit comprising the surgical bandage of claim 1 and a source of fluid tissue adhesive.

14. The kit of claim 13, wherein the tissue adhesive is enhanced viscosity cyanoacrylate.

15. The surgical bandage of claim 6 wherein the end segments and central bridging segment are formed from different material.
